SEQUENCE LISTING (3 sequences)

SEQ ID NO: 1 - protein, 36 aa, Artificial Sequence
  Synthetic
  MOD_RES (36)..(36): AMIDATION of the C-terminus residue
YPIKPEAPGEDASPEELNRYYASLRHYLNLVTRQRY

SEQ ID NO: 2 - protein, 34 aa, Artificial Sequence
  Synthetic
  MOD_RES (34)..(34): AMIDATION of the C-terminus residue
IKPEAPGEDASPEELNRYYASLRHYLNLVTRQRY

SEQ ID NO: 3 - protein, 34 aa, Artificial Sequence; identical to SEQ ID NO: 2
  Synthetic
  MOD_RES (1)..(2): Ile (position 1) is substituted by FMS, Lys (position 2) is substituted by epsilon-FMS
  MOD_RES (34)..(34): AMIDATION of the C-terminus residue

(19) United States
(12) Patent Application Publication
(10) Pub. No.: US 2007/0027073 A1
(43) Pub. Date: Feb. 1, 2007

(54) Long-acting derivatives of pyy agonists

(76) Inventors: Menachem Rubinstein, Rehovot (IL); Yoram Shechter, Rehovot (IL); Matityahu Fridkin, Rehovot (IL)

Correspondence Address: BROWDY AND NEIMARK, P.L.L.C.;624 NINTH STREET, NW, SUITE 300, WASHINGTON, DC, 20001-5303, US

(21) Appl. No.: 10/552,591

(22) PCT Filed: Apr. 8, 2004
(86) PCT No.: PCT/IL04/00320

§ 371 Date: Aug. 2, 2006

(30) Foreign Application Priority Data
Apr. 8, 2003 (US) 60460820

(51) Int. Cl. A61K 38/22; C07K 14/575

(52) U.S. Cl. 514/12; 530/324

(57) ABSTRACT

The invention provides a PYY agonist derivative of the formula: (X)ₙ-Z, wherein X is a radical 9-fluorenylmethoxy-carbonyl (Fmoc) or 2-sulfo-9-fluorenyl-methoxycarbonyl (FMS), Z is the residue of a PYY agonist linked to the radical X through an amino or hydroxyl group, and n is 1 to 3, or a pharmaceutically acceptable salt thereof, for reducing food intake and treatment of a disease, condition or disorder that can be alleviated by reduction of food intake such as obesity, hypertension, dyslipidemia, cardiovascular risk, eating disorder, insulin-resistance, or diabetes mellitus.

### FIELD OF THE INVENTION

The present invention relates to novel long-acting derivatives of PYY agonists that, following administration, are capable of undergoing spontaneous chemical transformation in the body from an inactive form into a biologically active PYY agonist, and particularly to derivatives of PYY agonists bearing a functional group sensitive to mild basic conditions, and to pharmaceutical compositions comprising them for reducing food intake and for treating diseases or disorders such as obesity.

Abbreviations: Fmoc: 9-fluorenylmethoxycarbonyl; FMS: (2-sulfo)-9-fluorenylmethoxycarbonyl; FMS-OSu: FMS N-hydroxysuccinimide ester; (FMS)₂-PYY₃₋₃₆: the peptide PYY₃₋₃₆ having two FMS moieties covalently attached to amino groups of PYY₃₋₃₆; HPLC: high-performance liquid chromatography.

### BACKGROUND OF THE INVENTION

The term obesity implies an excess of adipose tissue relative to lean body mass. It is best viewed as any degree of excess adiposity that imparts a health risk. Obesity results from a greater consumption of energy than is used by the body. As this energy is stored, fat cells enlarge and increase in number, producing the characteristic pathology of obesity.

Obesity is associated with important psychological and medical morbidities and represents a known risk for disorders and diseases such as hypertension; dyslipidemia; type 2 diabetes; coronary heart disease; stroke; gallbladder disease; osteoarthritis; sleep apnea and other respiratory problems; and endometrial, breast, prostate, and colon cancers.

Treatment of obesity remains a problem. Except for exercise, diet and food restriction, there is currently no convincing pharmacological treatment for effective reduction of body weight. Plain diet usually fails due to poor compliance and, when terminated, patients usually return to their pre-diet weight. One approved drug, Orlistat (Xenical), reduces fat adsorption through the gut by about one third, but it is poorly effective and has several side effects. An alternative pharmacological approach is based on appetite suppressants, but these medications are, in general, modestly effective. Some antidepressant medications have been studied as appetite suppressant medications, but were not found effective. Amphetamines and closely-related compounds are not recommended for use in the treatment of obesity due to their potential for abuse and dependence.

The peptides called NPY, PYY, and PP are hormones often said to belong to the pancreatic polypeptide family. Neuropeptide Y (NPY) is the most abundant peptide in central and peripheral nervous system in mammals. It stimulates food intake, affects blood pressure, enhances memory retention, and affects circadian rhythms. Human pancreatic polypeptide (PP), as isolated from the pancreas, has 36 amino acid residues with an amidated C-terminal tyrosine. PP is released into the plasma when stimulated by the ingestion of food and inhibits the stimulation of gastric and pancreatic exocrine secretions. The presence of the C-terminal tyrosine amide seems to be required for biological activity. A related peptide was discovered in extracts of intestine and named Peptide YY (PYY) because of its N- and C-terminal tyrosine (Y) residues.

The hypothalamic family of neuropeptide Y (NPY) receptors plays a major role in regulating satiety and food intake (Schwartz, 2000). The putative inhibitory Y2 pre-synaptic receptor (Y2R) is expressed in the arcuate nucleus, which is accessible to local and peripheral agonists of the NPY family (Broberger et al., 1997; Kalra et al., 1999). One such Y2R agonist is peptide YY₃₋₃₆ (PYY₃₋₃₆), which is released from the gastrointestinal tract post-prandially in proportion to the caloric content of a meal (Pedersen-Bjergaard et al., 1996; Adrian et al., 1985; Grandt et al., 1994). Recently, it was demonstrated that peripheral administration of PYY₃₋₃₆ inhibits food intake in humans, mice and rats and reduces weight gain in rats (Batterham et al., 2002, 2003; WO 02/47712). Thus, infusion of PYY₃₋₃₆ to reach the normal post-prandial circulatory concentrations of this peptide lead to a peak in serum PYY₃₋₃₆ within 15 min, followed by a rapid decline to normal levels within 30 min. Despite this rapid clearance, administration of PYY₃₋₃₆ to fasting individuals decreases their appetite and reduces food intake by 33% within a 12 h period following PYY₃₋₃₆ administration. Furthermore, no compensatory food intake occurs over the next 12 h (Batterham et al., 2002). Therefore, PYY₃₋₃₆ may find a clinical use in treatment of obesity and its associated disorders, including type II diabetes mellitus and cardiovascular diseases (Schwartz and Morton, 2002).

PYY and PYY agonists such as the fragment PYY₃₋₃₆ have been recently disclosed in WO 02/47712 as potential drugs for treatment of obesity and for treating conditions or disorders which can be alleviated by reducing nutrient availability in a subject, e.g. hypertension, dyslipidemia, cardiovascular risk, eating disorder, insulin-resistance, obesity and diabetes merlitus. Peripheral injection of PYY₃₋₃₆ in rats inhibits food intake and reduces weight gain. In humans, infusion of normal posprandial concentrations of PYY₃₋₃₆ has been shown to significantly decrease appetite and to reduce food intake by 33% over 12 h following administration of PYY₃₋₃₆. However, this effect of PYY₃₋₃₆ was limited to the first 12 h as no difference in food intake was noticed between the PYY₃₋₃₆ and placebo groups at the next 12 h (Batterharn et al., 2002).

WO 98/05361 discloses a novel conceptual approach for generation of long-acting drugs by derivatizing a drug having at least one free amino, carboxyl, hydroxyl andior mercapto groups with a moiety that is highly sensitive to bases and is removable under mild basic conditions. The prodrug obtained is inactive but undergoes transformation into the active drug under physiological conditions in the body. Examples of said moieties are the radicals 9-fluorenylmethoxycarbonyl (Fmoc) and 2-sulfo-9-fluorenylmethoxycarbonyl (FMS). According to this concept, Fmoc and FMS derivatives of of peptidic drugs such as insulin and human growth hormone, as well as of non-peptidic drugs such as propanolol, cephalexin and piperacillin (WO 98/05361), and of cytokines (WO 02/36067) and of enkephalin, doxorubicin, amphotericin B, gentamicin and gonadotropin releasing hormone (GnRH) (WO 02/7859) have been described.

It would be highly desirable to provide a derivative of a PYY agonist that has a longer circulatory half-life in the body.

### SUMMARY OF THE INVENTION

The present invention relates to a PYY agonist derivative of the formula:
(X)ₙ-Z
wherein X is a 9-fluorenylmethoxycarbonyl (Fmoc) or 2-sulfo-9-fluorenyl-methoxycarbonyl (FMS) radical, Z is the residue of a PYY agonist linked to the radical X through an amino or hydroxyl group, and n is 1 to 3.

A “PYY agonist” as defmed herein refers to a molecule that has a PYY- or PYY₃₋₃₆-like biological activity such as reducing food intake in mammals, and acts by a mechanism similar to that of PYY and PYY₃₋₃₆, for example by binding to the Y2 receptor. The PYY agonist is preferably an agonist specific for the Y2 receptor and is preferably a peptide containing, at a minimum, the sequence of amino acids 25-36 of PYY, most preferably, the sequence 3-36 of PYY.

In one embodiment of the invention, the PYY agonist is the 36-mer peptide PYY of the sequence represented by [SEQ ID NO.: 1]:

YPIKPEAPGEDASPEELNRYYASLRHYLNLVTRQRY-NH₂

In a preferred embodiment of the invention, the PYY agonist is the peptide PYY[3-36] of the sequence represented by [SEQ ID NO.: 2]:

IKPEAPGEDASPEELNRYYASLRHYLNLVTRQRY-NH₂

In a most preferred embodiment of the invention the radical X is FMS, Z is PYY₃₋₃₆ and n is 2, namely the derivative (FMS)₂-PYY₃₋₃₆, of the sequence represented by [SEQ ID NO.: 3]:

FMS-Ile-Lys(e-FMS)-Pro-Glu-Ala-Pro-Gly-Glu-Asp-
Ala-Ser-Pro-Glu-Glu-Leu-Asn-Arg-Tyr-Try-Ala-Ser-
Leu-Arg-His-Tyr-Leu-Asn-Leu-Val-Thr-Arg-Gln-Arg-
Tyr-NH₂

The invention further relates to a pharmaceutical composition comprising a PYY agonist derivative of the formula (X)ₙ-Z and a pharmaceutically acceptable carrier, particularly for reduction of food intake and for the treatment of diseases, conditions or disorders which can be alleviated by reduction of food intake.

The invention still further relates to a method of treatment of a disease, condition or disorder which can be alleviated by reduction of food intake which comprises administering to an individual a PYY agonist derivative of the formula (X)ₙ-Z, in an amount sufficient for reduction of food intake by said individual and consequent alleviation of said disease, condition or disorder.

### BRIEF DESCRIPTION OF THE DRAWING

FIG. 1 shows mass spectrometric analysis of (FMS)₂-PYY₃₋₃₆. Three peaks are apparent: 4350.96 corresponds to a monosubstituted FMS-PYY₃₋₃₆; 4653.77, corresponds to (FMS)₂-PYY₃₋₃₆ and 4675.91 corresponds to Na(FMS)₂-PYY₃₋₃₆.

### DETAILED DESCRIPTION OF THE INVENTION

The present invention provides novel PYY agonist derivatives in which one or more amino or hydroxyl groups of the PYY agonist is substituted with a radical Fmoc or FMS. These fumctional groups are sensitive to bases and are removable under mild basic conditions, e.g., under physiological conditions.

The derivatives of PYY agonists of the invention are prodrugs essentially lacking biological activity. They are metabolically stable and their rate of degradation and clearance in vivo is significantly lower than that of their corresponding PYY agonists. However, following administration, said prodrugs are capable of spontaneous gradual conversion to the corresponding PYY agonist and exhibit, therefore, an augmented bioavailability and are useful as long-acting PYY agonists.

In one embodiment of the invention, the radical X is Fmoc or FMS and the PYY agonist Z is the peptide PYY of SEQ ID NO: 1, thus obtaining the derivatives selected from the group consisting of Fmoc-PYY, (Fmoc)₂-PYY, (Fmoc)₃-PYY, FMS-PYY, (FMS)₂-PYY and (FMS)₃-PYY.

In another preferred embodiment of the invention, the radical X is Fmoc or FMS and the PYY agonist Z is the peptide PYY₃₋₃₆, thus obtaining the derivatives selected from the group consisting of Fmoc-PYY₃₋₃₆, (Fmoc)₂-PYY₃₋₃₆, (Fmoc)₃-PYY₃₋₃₆, FMS-PYY₃₋₃₆, (FMS)₂-PYY₃₋₃₆, and (FMS)₃-PYY₃₋₃₆.

In a most preferred embodiment, the long-acting derivative of the invention is (FMS)₂-PYY₃₋₃₆, in which one FMS radical is linked to the α-amino group of the N-terminal residue of PYY₃₋₃₆, and a second FMS radical is linked to the ε-amino group of the lysine (K) residue at position 2 of PYY₃₋₃₆, as herein represented by SEQ ID NO:3.

The PYY agonist derivatives according to the invention may be obtained by reacting the PYY agonist with excess of 9-fluorenylmethyl N-hydroxy-succinimide ester (Fmoc-OSu) or 2-sulfo-9-fluorenylmethyl N-hydroxy-succinimide ester (FMS-OSu), reagents that are very specific for amino groups, or with 9-fluorenylmethoxycarbonyl chloride (Fmoc-Cl), that reacts with, and covalently attaches to, amino and hydroxyl radicals.

Alternatively, the PYY agonist derivatives according to the invention may be obtained by direct peptide synthesis, using suitably derivatized lysine and the suitably derivatized N-terminal amino acid. Examples of these suitably derivatized amino acids are N-alpha-tBoc-N-epsilon-FMS-L-lysine and N-alpha-FMS-L-glutamic acid gamma t-butyl ester. Such derivatized amino acids are incorporated into the peptide sequence and following acid de-protection and cleavage from the resin, will yield (FMS)₂-PYY₃₋₃₆ with a free carboxyl at its N-terminus.

In a further alternative, the peptide PYY₅₋₃₆ is first prepared by solid phase automatic synthesis followed by manual addition of the suitably derivatized FMS-Lys and and FMS-Ile residues at the N-terminus, respectively.

According to the present invention (FMS)₂-PYY₃₋₃₆ was prepared by reacting one equivalent of PYY₃₋₃₆ in phosphate buffer pH 7.2, 0.1 M with 7 equivalents of FMS-OSu dissolved in dry dimethyl formamide (DMF) for 2 h. The reaction mixture was then subjected to extensive dialysis against distilled water at pH 6 and the retained fraction was saved for firrther study. Electrospray mass spectrometry of the retained fraction reveals a major signal at molecular mass 4654, corresponding to the formula (FMS)₂-PYY₃₋₃₆. A minor signal at molecular mass 4676 represents a sodium salt of (FMS)₂-PYY₃₋₃₆. Another minor signal at molecular mass 4351 represents a mono-substituted product FMS-PYY₃₋₃₆. Altogether, the proportion of (FMS)₂-PYY₃₋₃₆ represented 85% of the material and the remainder was mono-substituted FMS-PYY₃₋₃₆. Further purification of (FMS)₂-PYY₃₋₃₆ may be obtained by conventional chromatographic methods, e.g., reversed-phase HPLC.

To evaluate the efficacy of (FMS)₂-PYY₃₋₃₆ as modulator of food intake, it was studied in a re-feeding model. Briefly, normal C57BL/6 male mice were subjected to a starvation period of 24 h with unrestricted supply of drinking water. At different time points during the starvation the mice were injected intra-peritoneally with either saline, PYY₃₋₃₆ or (FMS)₂-PYY₃₋₃₆. At the end of the starvation period, the mice were presented with pre-weighted supply of standard chow and the amount of food consumed during the first 2 h was recorded. Each study group consisted of 10 mice and the amount of food consumed was measured per 10 mice. When mice were injected with 2.5 μg of PYY₃₋₃₆ in 0.1 ml saline at time 24 h (immediately before providing the food), there was a 28% reduction of food intake as compared with a control group of mice injected with saline. When mice were injected with 2.5 μg of PYY₃₋₃₆ in 0.1 ml saline at time 23 h 45 min (15 min. before providing the food), there was a 40% reduction of food intake as compared with a control group of mice injected with saline. The improved response to PYY₃₋₃₆ in the second experiment was due to higher food consumption in the group injected with saline at 23 h 45 min as compared with the group injected with saline at 24 h. Apparently, the stress imposed by the injection itself has also reduced food consumnption. In another study, mice were injected with 1 μg of PYY₃₋₃₆ at time 23 h, 45 min. and the amount of food consumed was reduced by 31% as compared with a saline-injected group. Thus, a dose-response was obtained.

To establish the duration of response to unmodified PYY₃₋₃₆, 2.5 μg of PYY₃₋₃₆ were injected to starving mice at time 19 h (5 h before providing the food). No effect on food intake was seen, as compared with saline control. The efficacy of the (FMS)₂-PYY₃₋₃₆ prodrug was then tested. Mice were injected with 5 μg of (FMS)₂-PYY₃₋₃₆ per mouse at time 23 h, 45 min. The amount of food consumed was 90% of that consumed by a saline-injected group. This experiment indicated that (FMS)₂-PYY₃₋₃₆ probably lacked biological activity prior to being hydrolyzed to active PYY₃₋₃₆. In another experiment, mice were injected with 50 μg of (FMS)₂-PYY₃₋₃₆ at time 18 h, namely 6 h before re-feeding. This time, the amount of food consumed was reduced by 49% as compared with a saline-injected group. In another study, mice were injected with 20 μg of (FMS)₂-PYY₃₋₃₆ at time 5 h, namely 19 h before re-feeding. This time, the amount of food consumed at time 24-26 h was reduced by 26% as compared with a saline-injected group. These experiments indicate that (FMS)₂-PYY₃₋₃₆ is an inactive long-acting prodrug that produces active PYY₃₋₃₆ following administration into mice. It is concluded that (FMS)₂-PYY₃₋₃₆ reduces food intake even when given 19 h before food. This is in contrast with unmodified PYY₃₋₃₆, whose activity is rapidly eliminated and is not detectable 5 h after administration. The results obtained according to the invention indicate that long-acting PYY agonist derivatives such as (FMS)₂-PYY₃₋₃₆ have utility in reducing food intake, and can be used as therapeutics to treat diseases, conditions or disorders that benefit from reduced food intake, such as obesity.

Also included in the scope of the invention are pharmaceutically acceptable salts of the PYY agonist derivatives of the invention. As used herein, the term “salts” refers to both salts of carboxyl groups and to acid addition salts of amino groups of the peptide molecule. Salts of a carboxyl group may be formed by means known in the art and include inorganic salts, for example, sodium, calcium, ammonium, ferric or zinc salts, and the like, and salts with organic bases such as those formed for example, with amines, such as triethanolamine, arginine, or lysine, piperidine, procaine, and the like. Acid addition salts include, for example, salts with mineral acids such as, for example, hydrochloric acid or sulfuc acid, and salts with organic acids, such as, for example, acetic acid or oxalic acid. Such salts may be preferably used to modify the pharmaceutical properties of the peptide insofar as stability, solubility, etc., are concerned.

In another aspect, the present invention relates to pharmaceutical compositions comprising a PYY agonist derivative of the invention or a pharmaceutically acceptable salt thereof and a pharmaceutically acceptable carrier.

The carrier must be “acceptable” in the sense of being compatible with the active ingredient(s) of the formulation (and preferably, capable of stabilizing peptides) and not deleterious to the subject to be treated.

The formulations may conveniently be presented in unit dosage form and may be prepared by any of the methods well known in the art of pharmacy, for example as described in Remington: The Science and Practice of Pharmacy, A.R. Gennaro, ed., 20th edition, 2000. All methods include the step of bringing the active ingredient(s) into association with the carrier which constitutes one or more accessory ingredients.

Any suitable route of administration of the PYY agonist derivatives to humans is envisaged by the invention, for example via conventional injectable, intramuscular, intravenous, subcutaneous, intranasal and transdermal administration.

The pharmaceutical compositions of the invention are useful for reduction of food intake and for the treatment of diseases, conditions or disorders that can be alleviated by reduction of food intake, such as, but not limited to, obesity, hypertension, dyslipidemia, cardiovascular risk, insulin-resistance, or diabetes mellitus (particularly diabetes of type II).

In another aspect, the invention relates to a method for reduction of food intake or for treatment of a disease, condition or disorder that can be alleviated by reduction of food intake which comprises administering to an individual in need thereof an effective amount of a PYY agonist derivative of the invention. Any disease, condition or disorder known today or to be discovered in the future that can be can be alleviated by reduction of food intake such as, but not limited to, obesity, hypertension, dyslipidemia, cardiovascular risk, eating disorder, insulin-resistance, and diabetes mellitus, is envisaged according to the invention for treatment with the respective derivative of the PYY agonist according to the present invention.

The invention further relates to a method of inducing weight loss in an individual comprising administering to said individual a therapeutically effective amount of a PYY agonist derivative of the invention, preferably,(FMS)₂-PYY₃₋₃₆, or a pharmaceutically acceptable salt thereof.

The invention will now be illustrated by the following non-limiting Examples.

### EXAMPLES

### Example 1

### Preparation of (BMS)2-PYY₃₋₃₆

PYY₃₋₃₆ was prepared by solid-phase peptide synthesis or alternatively purchased from Bachem AG, Bubendorf, Switzerland.

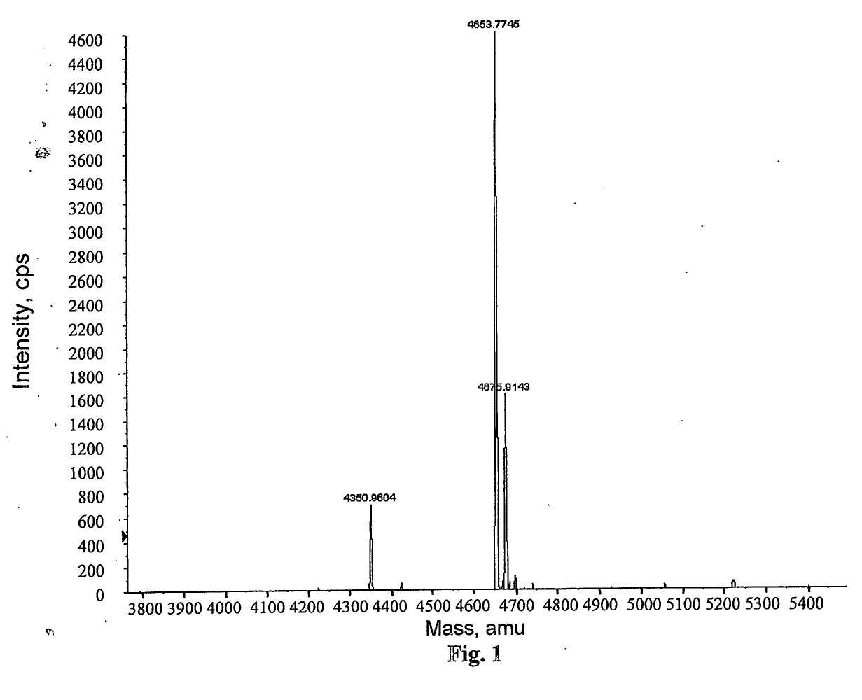

Procedure I. PYY₃₋₃₆ was dissolved in distilled water and its concentration was determined to be 0.84 mg/ml at OD₂₈₀ (ε=6400). A solution of PYY₃₋₃₆ (0.4 ml) was then mixed with 0.1 ml of phosphate buffer pH 7.2, 0.1 M. FMS-OSu (prepared as described in WO 02/36067), 240 μg (7 molar equivalents), dissolved in dry dimethyl formamide (DMF), was added and the mixture stirred for 2 h. The reaction mixture was then subjected to extensive dialysis at 4° C. against distilled water at pH 6. At the end of dialysis, the retained volume was 1.55 ml and the calculated concentration was 0.2 mg/ml. Electrospray mass spectrometry of the retained fraction revealed a major signal at molecular mass 4654, corresponding to the formula (FMS)₂-PYY₃₋₃₆. A minor signal at molecular mass 4676 represents a sodium salt of (FMS)₂-PYY₃₋₃₆. Another minor signal at molecular mass 4351 represents a monosubstituted product FMS-PYY₃₋₃₆. Altogether, the proportion of (FMS)₂-PYY₃₋₃₆ represented 85% of the material and the remainder was mono-substituted FMS-PYY₃₋₃₆ or trisubstituted (FMS)₃-PYY₃₋₃₆ (FIG. 1). The title compound was further purified by applying the mixture onto a C18 reversed-phase HPLC column and resolving the two products by a gradient of acetonitrile in 0.1% aq. trifluoroacetic acid. (FMS)2-PYY₃₋₃₆ elutes after the monosubstituted FMS-PYY₃₋₃₆.

Procedure II. According to this procedure, first the peptide PYY₅₋₃₆ is prepared by solid phase automatic procedure and then the two FMS groups at positions 2 and 1 are introduced manually while adding the Lys and Ile residues, respectively. Thus, synthesis of polymer-bound PYY₅₋₃₆ is achieved by solid phase automatic procedure employing an ABIMED AMS422 synthesizer (ABIMED, Lanegenfeld, Germany) using the commercially available protocols via the Fmoc-strategy. All protected amino acid derivatives as well as the polymeric support 4-([2′, 4′-dimethoxyphenyl]-Fmoc-amino-ethyl) phenoxy resin (Rink Amid Resin) are purchased from Nova, Switzerland. Coupling is achieved by PyBOP (benzotriazolyl-N-oxy-tris(dimethylamino) phosphonium hexafluorophosphate.

The free α-amino group of Pro-5 is coupled manually and using the same steps performed automatically, to α-Mtt-Lys(ε-FMS)-OH. The Mtt-protecting group (4-methyltrityl) is then removed by treatment with TFA:TES:DCM (2:5:93; v:v:v) for 30 min (6×5 min) and the free α-amino group of Lys-4 is coupled manually with FMS-Ile. After completion of coupling, final cleavage of the peptide from the resin along with side-chain deprotection is achieved by treatment with a mixture of TFA:water:TES (95:2.5:2.5; v:v:v) for 2 h. The cleaved peptide is precipitated with ice cold tert-butylmethyl ether and centrituged. The solution is decanted and the pellet is dissolved in water and lyophilized to yield a white powder. Purification of the crude FMS-peptide is performed as described above.

### Example 2

### PYY[3-36] Reduces Food Intake in a Mouse Re-feeding Model

The efficacy of (FMS)2-PYY₃₋₃₆ as a modulator of food intake was studied using a re-feeding model. A group of 10 normal C57BL/6 male mice at the age of 9 weeks were subjected to starvation for a period of 24 h with unrestricted supply of drining water. At the end of this period, the mice were injected intra-peritoneally with either 0.1 ml saline or 2.5 μg PYY₃₋₃₆ dissolved in 0.1 ml saline per mouse. The mice were then immediately presented with pre-weighted supply of standard chow and the amount of food consumed during the first 2 h was recorded. Each study group consisted of 10 mice and the amount of food consumed as reported in this example was per 10 mice. The amount of chow consumed by the group of 10 saline-injected control mice was 9.9 g. The amount of chow consumed by the group of 10 PYY₃₋₃₆-injected mice was 7.1 g. Hence, there was a 28% reduction of food intake following administration of PYY₃₋₃₆ as compared with a control group of mice injected with saline.

### Example 3

### PYY₃₋₃₆ Reduces Food Intake in an Improved Mouse Re-feeding Model

In order to assess whether the handling of the mice and the act of injection itself resulted in stress-induced loss of appetite, which could reduce the difference in food intake between the control and the PYY₃₋₃₆-injected mice, the experiment was slightly modified. Two groups of 10 normal male C57BL/6 mice at the age of 10 weeks were subjected to starvation for a period of 24 h, with unrestricted supply of drinking water. At time 23 h 45 min, one group of mice was injected intraperitoneally with 0.1 ml saline and the second group was injected with 2.5 μg PYY₃₋₃₆ dissolved in 0.1 ml saline per mouse. The mice were presented at 24 h with a pre-weighted supply of standard chow and the amount of food consumed during the first 2 h was recorded. The amount of chow consumed by the group of 10 saline-injected control mice was 10.9 g. The amount of chow consumed by the group of 10 PYY₃₋₃₆-injected mice was 6.5 g. Hence, there was a 40% reduction of food intake following administration of PYY₃₋₃₆ as compared with the control group of mice injected with saline.

### Example 4

### Determining the Duration of the Effect of PYY₃₋₃₆ on Food Intake

Two groups of 10 normal male C57BL/6 mice at the age of 12 weeks were subjected as before to starvation for a period of 24 h with unrestricted supply of drinking water. At time 19 h, one group of mice was injected intra-peritoneally with 0.1 ml saline and the second group was injected with 2.5 μg PYY₃₋₃₆ dissolved in 0.1 ml saline per mouse. The mice were presented at 24 h with a pre-weighted supply of standard chow and the amount of food consumed during the first 2 h was recorded. The amount of chow consumed by the group of 10 saline-injected control mice was 11.2 g. The amount of chow consumed by the group of 10 PYY₃₋₃₆-injected mice was 11.4 g. Hence, there was no effect on food intake as compared with saline control. Therefore, when PYY₃₋₃₆ is administered at a dose of 2.5 μg per mouse, its effect on food intake is lost after 5 h.

### Example 5

### (FMS)₂-PYY₃₋₃₆ has a Small Immediate Effect on Food Intake

Two groups of 10 normal male C57BL/6 mice at the age of 12 weeks were subjected to starvation for a period of 24 h with unrestricted supply of dinking water. At time 23 h 45 min, one group of mice was injected intra-peritoneally with 0.1 ml saline. The second group was injected with 3 microgram (FMS)₂-PYY₃₋₃₆ dissolved in 0.1 ml saline per mouse. The mice were presented at 24 h with a pre-weighted supply of standard chow and the amount of food consumed during the first 2 h was recorded. The amount of chow consumed by the group of 10 saline-injected control mice was 11 g. The amount of chow consumed by the group of 10 (FMS)₂-PYY₃₋₃₆-injected mice was 9.9 g. Hence, there was a relatively small effect (10%) of (FMS)₂-PYY₃₋₃₆ on food intake. This effect may be attributed to hydrolysis of (FMS)₂-PYY₃₋₃₆ into active PYY₃₋₃₆ during the feeding period of 2 h. It is therefore concluded that (FMS)₂-PYY₃₋₃₆ probably lacks biological activity before being hydrolyzed into active PYY₃₋₃₆.

### Example 6

### Regeneration of Biologically Active PYY₃₋₃₆ by Mild Hydrolysis of (FMS)₂-PYY₃₋₃₆

(FMS)₂-PYY₃₋₃₆ was dissolved (0.1 mg/ml) in 0.1 M NaHCO₃ (pH=8.5) and the solution was kept in a sealed test tube for 42 h at 37° C. The reaction mucure was then subjected to extensive dialysis against water. Two groups of 10 normal male C57BL/6 mice at the age of 12 weeks were subjected to starvation for a period of 24 h with unrestricted supply of drinking water. At time 23 h 45 min, one group of mice was injected intraperitoneally with 0.1 ml of saline. The second group was injected with the retained fraction of the dialysis step (3 μg hydrolyzed (FMS)₂-PYY₃₋₃₆ in 0.1 ml saline per mouse. The mice were presented at 24 h with a pre-weighted supply of standard chow and the amount of food consumed during the first 2 h was recorded. The amount of chow consumed by the group of 10 saline-injected control mice was 11 g. The amount of chow consumed by the group of 10 mice injected with hydrolyzed (FMS)₂-PYY₃₋₃₆ was 6.6 g. Hence, there was a 40% reduction in food intake, indicating that (FMS)₂-PYY₃₋₃₆ was hydrolyzed into active PYY₃₋₃₆.

### Example 7

### Long-acting Effect of (FMS)₂-PYY₃₋₃₆ on Food Intake

Two groups of 10 normal male C57BL/6 mice at the age of 12 weeks were subjected to starvation for a period of 29 h with unrestricted supply of drinking water. At time 23 h, one group of mice was injected intra-peritoneally with 0.1 ml saline. The second group was injected with 50 μg (FMS)₂-PYY[3-36] dissolved in 0.1 ml saline. The mice were presented at 29 h with a pre-weighted supply of standard chow and the amount of food consumed during the first 2 h was recorded. The amount of chow consumed by the group of 10 saline-injected control mice was 12 g. The amount of chow consumed by the group of 10 (FMS)₂-PYY[3-36]-injected mice was 5.9 g. Thus, the amount of food consumed was reduced by 51% as compared with a saline-injected control group. These experiments indicate that (FMS)₂-PYY[3-36] is a long-acting prodrug that has a dramatic effect on food uptake by hydrolyzing into active PYY[3-36] in mice.

This experiment was repeated using a smaller dose (20 μg) of (FMS)₂-PYY[3-36] that was given 4 h before re-feeding at 24 h. This time the reduction in food intake was still significant. The group of 10 mice receiving saline ate 12.3 g, whereas the group receiving 20 μg of (FMS)₂-PYY[3-36] ate 7.4 g, namely, a 40% reduction in food intake.

### Example 8

### Longer-acting Effect of (FMS)₂-PYY[3-36] on Food Intake

Two groups of 10 normal male C57BL/6 mice at the age of 12 weeks were subjected to starvation for a period of 24 h with unrestricted supply of drinking water. At time 45 h, one group of mice was injected intraperitoneally with 0.1 ml saline. The second group was injected with 20 μg (FMS)₂-PYY[3-36] dissolved in 0.1 ml saline. The mice were presented at 24 h with a pre-weighed supply of standard chow and the amount of food consumed during the first 2 h was recorded. The amount of chow consumed by the group of 10 saline-injected control mice was 11.2 g. The amount of chow consumed by the group of 10 (FMS)₂-PYY[3-36]-injected mice was 8.3 g. Thus, the amount of food consumed was reduced by 26% as compared with the saline-injected control group. These experiments indicate that (FMS)₂-PYY[3-36] is a long-acting prodrug that has a dramatic effect on food uptake by hydrolyzing into active PYY[3-36] in mice.

### REFERENCES

Adrian T E, Ferri G L, Bacarese-Hamilton A J, Fuessl H S, Polak J M, Bloom S R: Human distribution and release of a putative new gut hormone, peptide YY. Gastroenterol. 89:1070-1077, 1985

Batterham R L, Cowley M A, Small C J, Herzog H, Cohen M A, Dakin C L, Wren A M, Brynes A E, Low M J, Ghatei M A, Cone R D, Bloom S R: Gut hormone PYY₃₋₃₆ physiologically inhibits food intake. Nature 418:650-654, 2002

Batterham R L, Cohen M A, Ellis S M, Le Roux C W, Withers D J, Frost G S, Ghatei M A, Bloom S R: Inhibition of food intake in obese subjects by peptide YY3-36. N Engl J Med 349:941-948, 2003

Broberger C, Landry M, Wong H, Walsh J N, Hokfelt T: Subtypes of Y1 and Y2 of the neuropeptide Y receptor are respectively expressed in pro-opiomelanocorin and neuropeptide-Y containing neurons of the rat hypothalamic arcuate nucleus. Neuroendocrinol. 66:393-408, 1997

Grandt D, Schimiczek M, Belinger C, Layer P, Goebell H, Eysselein V E, Reeve J R J: Two molecular forms of peptide YY (PYY) are abundant in human blood: characterization of a radioimmunoassay recognizing PYY 1-36 and PYY 3-36. Regul. Pept. 51:151-159, 1994

Kalra S P, Dube M G, Pu S, Xu B, Horvath T L, Kalra P S: Interacting appetite-regulating pathways in the hypothalamic regulation of body weight. Endocr. Rev. 20:68-100, 1999

Pedersen-Bjergaard U, Host U, Kelbaek H, Schifter S, Rehfeld J F, Faber J, Christensen N J: Influence of meal composition on postprandial peripheral plasma concentrations of vasoactive peptides in man. Scand. J Clin. Lab. Invest. 56:497-503, 1996

Schwartz M W, Morton G J: Obesity: keeping hunger at bay. Nature 418:595-597,2002

Schwartz M W, Woods S C, Porte D J, Seeley R J, Baskin D G: Central nervous system control of food intake. Nature 404:661-671, 2000

1. A PYY agonist derivative of the formula:
(X)ₙ-Z
wherein X is 9-fluorenylmethoxycarbonyl (Fmoc) or 2-sulfo-9-fluorenyl-methoxycarbonyl (FMS), Z is the residue of a PYY agonist linked to the radical X through an amino or hydroxyl group, and n is 1 to 3, or a pharmaceutically acceptable salt thereof.

2. A PYY agonist derivative of claim 1, wherein the PYY agonist is PYY of the sequence represented by SEQ ID NO.: 1:
YPIKPEAPGEDASPEELNRYYASLRHYLNLVTRQRY-NH₂.

3. A PYY agonist derivative of claim 1, wherein the PYY agonist is PYY₃₋₃₆ of the sequence represented by SEQ ID NO.: 2:
IKPEAPGEDASPEELNRYYASLRHYLNLVTRQRY-NH₂.

4. The PYY agonist derivative of claim 1 selected from the group consisting of the derivatives herein designated Fmoc-PYY, (Fmoc)₂-PYY, (Fmoc)₃-PYY, FMS-PYY, (FMS)₂-PYY and (FMS)₃-PYY.

5. The PYY agonist derivative of claim 1 selected from the group consisting of the derivatives herein designated Fmoc-PYY₃₋₃₆, (Fmoc)₂-PYY₃₋₃₆, (Fmoc)₃-PYY₃₋₃₆, FMS-PYY₃₋₃₆, (FMS)₂-PYY₃₋₃₆, and (FMS)₃-PYY₃₋₃₆.

6. The PYY agonist derivative herein designated (FMS)₂-PYY₃₋₃₆, of the sequence represented by SEQ ID NO.: 3.

7. A pharmaceutical composition comprising a PYY agonist derivative according to claim 1, and a pharmaceutically acceptable carrier.

8. A pharmaceutical composition comprising (FMS)₂-PYY₃₋₃₆ and a pharmaceutically acceptable carrier.

9-16. (canceled)

17. A method for reduction of food intake which comprises administering to an individual in need an effective amount of a PYY agonist derivative of claim 1.

18. A method for treatment of a disease, condition or disorder that can be alleviated by reduction of food intake which comprises administering to an individual in need an effective amount of a PYY agonist derivative of claim 1.

19. The method according to claim 18 wherein said disease or disorder is obesity.

20. The method according to claim 18 wherein said disease or disorder is hypertension, dyslipidemia, cardiovascular risk, eating disorder, insulin-resistance, or diabetes mellitus.

21. A method of treating obesity in an individual comprising administering to said individual a therapeutically effective amount of (FMS)₂-PYY₃₋₃₆, or a pharmaceutically acceptable salt thereof.

22. A method of inducing weight loss in an individual comprising administering to said individual a therapeutically effective amount of a PYY agonist derivative of claim 1 or a pharmaceutically acceptable salt of said compound.

23. A method according to claim 22 wherein said PYY agonist derivative is (FMS)₂-PYY₃₋₃₆, or a pharmaceutically acceptable salt thereof.
